(19) United States
(12) Patent Application Publication
(10) Pub. No.: US 2025/0154572 A1
(43) Pub. Date: May 15, 2025

(54) INWARD FLUID DISPLACEMENT (IFD) IN ROTATIONAL MICROFLUIDIC DEVICE

(71) Applicant: University of Virginia Patent Foundation, Charlottesville, VA (US)

(72) Inventors: James P. Landers, Charlottesville, VA (US); Leah Michele Dignan, Charlottesville, VA (US); Michael Shane Woolf, Midlothian, VA (US)

(21) Appl. No.: 18/838,533

(22) PCT Filed: Feb. 17, 2023
(86) PCT No.: PCT/US2023/062823

§ 371 Date: Aug. 14, 2024

Related U.S. Application Data
(60) Provisional application No. 63/311,515, filed on Feb. 18, 2022.

(51) Int. Cl. C12Q 1/6844; B01L 3/00; C12Q 1/6806

(52) U.S. Cl. CPC C12Q 1/6844; B01L 3/502753; B01L 2200/10; B01L 2200/16; B01L 2300/0832; B01L 2300/0877; B01L 2300/10; B01L 2400/0409; B01L 2400/0622; C12Q 1/6806

(57) ABSTRACT

Apparatus and techniques described herein can include or use a rotationally-driven microfluidic assembly. For example, a sample can be propelled to a sample recovery chamber from a sample chamber using a gas evolved from a reaction between the liquid reagent and a dry reagent. Such gas evolution can provide displacement of a sample liquid or other liquid in an inward direction, such as proximally toward a center of rotation. Such gas evolution can include features or reagents, or both, that are compatible with downstream nucleic acid amplification tests.

### CLAIM OF PRIORITY

This patent application claims the benefit of priority of Landers et al., U.S. Provisional Patent Application Ser. No. 63/311,515, titled “METHOD AND SYSTEM FOR RADIALLY-INWARD FLUID DISPLACEMENT ON CENTRIFUGAL MICROFLUIDIC DEVICES,” filed on Feb. 18, 2022 (Attorney Docket No. 02794-01), which is hereby incorporated by reference herein in its entirety.

### FIELD OF THE DISCLOSURE

This document pertains generally, but not by way of limitation, to microfluidic devices and related techniques for performing liquid transfer in such devices in support of analysis of a sample.

### BACKGROUND

Demand for sample-to-answer point-of-need (PoN) nucleic acid (NA) analysis platforms has increased dramatically, particularly in view of the COVID-19 pandemic. Microfluidic devices offer an approach for performing such analysis. In one approach, a centrifugally-driven device can be used, such as to assist in analysis of a raw sample. Broadly, NA analysis may be divided into two groups of operations, sample preparation (e.g., cell lysis and NA extraction (NAE)) and a second group of operations including amplification and detection. Relatively little consideration has been dedicated to translation of laboratory-based sample preparation methods to microfluidic devices, particularly centrifugally-driven “lab-on-a-disk” (LoaD) devices. In some approaches, NAE is performed, but generally, many otherwise portable microfluidic NA amplification tests remain tethered to separate laboratory instrumentation or lengthy manual sample preparation protocols.

### SUMMARY OF THE DISCLOSURE

The present inventors have recognized, among other things that various technology gaps hamper development of fully-integrated sample-to-answer PON NA LoaDs, particularly where on-disc lysis and NAE capabilities are provided. The present inventors have also recognized, among other things, that an enabling technology to enhance LoaD capability is inward fluid displacement (IFD), where a reagent or analyte is transferred toward a center-of-rotation (CoR). Such IFD can facilitate inclusion of more operations on-disc, such as facilitating on-disc NA amplification and detection.

Generally, disc-based systems use of rotational forces to direct flow, which confers a high degree of compactness and portability that is distinct from analogous benchtop instrumentation. However, the present inventors have recognized, among other things, that using rotation as the sole motive fluidic force also creates operational limitations, because such rotation only provides unidirectional, radially outward flow relative to the CoR. Such flow constraints can place restrictions on microfluidic processing configurations. For example, once fluid reaches a disc periphery, little or no further on-board processing steps (unit operations) are possible. Accordingly, a count of sequential unit operations can be limited by LoaD radius and a footprint of the requisite microfluidic architecture (e.g., chambers, channels, valves, or other structures). To address such challenges, the present inventors have recognized that returning fluid from the disc periphery to a location nearer the CoR via IFD could facilitate integration of a wider range of unit operations on-disc. By allowing for performance of more sequential unit operations, IFD permits increased assay complexity, lowering barriers to production of sample-to-answer LoaDs.

In an example, a microfluidic assembly can be configured for rotationally-driven operation, the microfluidic assembly comprising a hub region defining a center-of-rotation (CoR), a dry reagent region, a liquid reagent chamber fluidically isolated from the dry reagent region by a liquid reagent valve, the liquid reagent chamber located more proximally to the CoR as compared to the dry reagent region, a sample chamber fluidically coupled with the dry reagent region through a gas transfer channel, and a sample recovery chamber fluidically coupled with the sample chamber, the sample recovery chamber located more proximally to the CoR as compared to the dry reagent region and the sample chamber. The liquid reagent valve, when opened, can permit a liquid reagent from the liquid reagent to flow in a direction distally with respect to the CoR to the dry reagent region in response to rotation of the microfluidic assembly about the CoR. The gas transfer channel can be configured to convey a gas evolved from a reaction between the liquid reagent and a dry reagent in the dry reagent region to the sample chamber to propel at least a portion of a sample in the sample chamber to the sample recovery chamber.

In an example, processing of sample using a rotationally-driven microfluidic assembly can be performed, such as using a method comprising conveying a sample to a sample chamber of the microfluidic assembly from a sample inlet region by rotating the microfluidic assembly about a center of rotation (CoR) defined by a hub region, actuating a liquid reagent valve to fluidically connect a liquid reagent chamber with a dry reagent region of the microfluidic assembly, conveying a liquid reagent from the liquid reagent chamber to the dry reagent region by rotating the microfluidic assembly about the CoR, isolating the liquid reagent chamber from the dry reagent region by re-sealing the liquid reagent valve, and propelling the sample to a sample recovery chamber from the sample chamber using a gas evolved from a reaction between the liquid reagent and a dry reagent located in the dry reagent region, the gas conveyed to the sample chamber using a gas transfer channel from the dry reagent region. The sample recovery chamber can be located more proximally to the CoR as compared to the dry reagent region and the sample chamber, and the sample can be propelled at least in part inwardly toward the CoR by the gas.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates generally an example of a microfluidic assembly comprising a disc, the microfluidic assembly configured to support inward fluidic displacement (IFD).

FIG. 1B illustrates generally an example of a portion of the microfluidic assembly of FIG. 1A.

FIG. 1C illustrates generally an exploded view of layers and other structures that can form the microfluidic assembly of FIG. 1A.

FIG. 1D illustrates a section view of a portion of the microfluidic assembly of FIG. 1A.

FIG. 2A, FIG. 2B, FIG. 2C, and FIG. 2D illustrate various operations that can be used to fabricate a membrane structure or other portion of the microfluidic assembly of FIG. 1A.

FIG. 3A, FIG. 3B, FIG. 3C, FIG. 3D, and FIG. 3E show operations that can be used to provide a dry reagent region (e.g., a cup), such as can be packed with one or more dry reagents that can be used for gas generation.

FIG. 4 illustrates a structure and related operation of a valve, such as can be included as a portion of the microfluidic assembly of FIG. 1A and the detailed view shown in FIG. 1B.

FIG. 5 illustrates operations that can be performed, such as for sample extraction, using the microfluidic assembly of FIG. 1A and the structures shown in FIG. 1B.

FIG. 6A shows an illustrative example of a correlation between extracted sample volume and pixel count for evaluation of performance of sample extraction assisted by gas evolution in a microfluidic assembly as shown in FIG. 1A and using the structures shown in FIG. 1B.

FIG. 6B shows corresponding experimentally-obtained sample recovery percentages (by volume) corresponding to different acid-base neutralization reactions for gas generation.

FIG. 7A shows an illustrative example of a correlation between extracted sample volume and starting sample volume for evaluation of performance of sample extraction assisted by gas evolution in a microfluidic assembly as shown in FIG. 1A and using the structures shown in FIG. 1B.

FIG. 7B shows an illustrative example of a correlation between extracted sample volume and aging of a dry reagent for evaluation of performance of sample extraction assisted by gas evolution in a microfluidic assembly as shown in FIG. 1A and using the structures shown in FIG. 1B.

FIG. 8A shows an illustrative example of polymerase chain reaction (PCR) compatibility with a sample that has been displaced by gas evolution in a microfluidic assembly as shown in FIG. 1A and using the structures shown in FIG. 1B, versus a non-displaced sample, and controls.

FIG. 8B and FIG. 8C show illustrative examples of full 18-plex short tandem repeat (STR) profiles obtained from DNA lysed and displaced by gas evolution in a microfluidic assembly as shown in FIG. 1A and using the structures shown in FIG. 1B prior to amplification.

FIG. 9A, FIG. 9B. FIG. 9C, and FIG. 9D show illustrative examples comprising respective electropherograms of controls and samples obtained from DNA lysed and displaced by gas evolution in a microfluidic assembly as shown in FIG. 1A and using the structures shown in FIG. 1B.

FIG. 10 shows illustrative examples of gel image renderings of triplicate samples, indicating successful amplification of samples displaced by gas evolution in a microfluidic assembly as shown in FIG. 1A and using the structures shown in FIG. 1B, versus controls.

FIG. 11 shows illustrative examples colorimetric evaluation of LAMP-based detection on samples displaced by gas evolution in a microfluidic assembly as shown in FIG. 1A and using the structures shown in FIG. 1B, versus controls, and indicating successful detection of all samples in less than 45 minutes.

FIG. 12 illustrates generally an example of a microfluidic assembly comprising a disc, the microfluidic assembly configured to support inward fluidic displacement (IFD) for sample extraction along with downstream LAMP.

FIG. 13 illustrates generally an example comprising operations to perform LAMP, such as using a microfluidic assembly as shown in FIG. 12.

FIG. 14 illustrates generally an example showing proper delivery of a lysate to the center sample chamber (indicated by hue), while such a lysate is isolated from the NTC and (+) control chambers, such as using the operations shown in FIG. 13.

FIG. 15 illustrates generally a technique, such as a method, for propelling a sample to a sample recovery chamber, such as in a manner toward a center of rotation (CoR) using gas evolution.

### DETAILED DESCRIPTION

As mentioned above, rotationally-driven microfluidic disc devices may present various limitations, as a liquid within the device eventually ends up at a periphery of the device structure located distally with respect to a center of rotation (CoR). Various approaches can be used to achieve displacement of fluid in a direction proximally toward the CoR (e.g., providing “inward fluid displacement” (IFD)). For example, capillary action, hydraulic pressure, or pneumatic pressure can be used. Exploiting an interplay between capillary and centrifugal forces can provide a simple approach, because it requires no peripheral reagents or equipment. However, spontaneous, partial capillary refill generally depends on the hydrophilic character and surface energy of the disc materials, and such characteristics may vary substantially in polymeric devices.

Other IFD strategies may use internal hydraulic or pneumatic pressure changes that exceed outwardly-oriented centrifugally-generated pressure. However, such approaches can still present various challenges. For example, IFD can be driven by increasing thermal energy, and therefore pressure, such as experienced by air volumes on-board a disc device. However, portability of such an approach may be limited by reliance on external hardware (e.g., a heat source such as a halogen lamp, IR thermometer, or other apparatus), and bulk heating may damage sensitive reagents on-board the disc. In yet another approach, physical (not thermal) compression and expansion of on-disc air pockets can be used to drive IFD. For example, this can be achieved using a displacer fluid, the volume of which generally exceeds that of the sample solution.

In yet another approach, an immiscible displacer liquid may drive IFD of a sample through direct application of centrifugally-generated hydrostatic pressure. Such pressure-based approaches mentioned above are generally slow (>90 seconds for an operation) and generally involve use of large chambers to contain air or displacer fluids. Such chambers consume significant disc area. In yet other approaches, large volumes of working fluid and associated microfeatures are not needed. For instance, introducing dry compressed air into a microfluidic vent can drive IFD. However, such an approach can present drawbacks, such as being pulsatile or using bulky hardware (e.g., air tanks separate from the microfluidic assembly), and such an approach may present risks related to contamination.

In one approach, gas may be generated via on-disc electrolysis, but this approach generally requires a disc configuration with electrodes. The present inventors have recognized, among other things, that gas generation can be performed on or within a microfluidic assembly, such as including generation of carbon dioxide (CO₂). Self-contained IFD can be established using positive pressure generated from on-board acid-base neutralization to drive fluid toward the CoR. As shown and described herein, the present inventors have, among other things, developed apparatus and techniques that can provide gas evolution in a manner suppressing or inhibiting sample acidification resulting from gas dissolution, so that such dissolution and related liquid displacement does not hinder downstream processing. Accordingly, the approaches shown and described herein are compatible with nucleic acid amplification tests (NAATs) and other related processing.

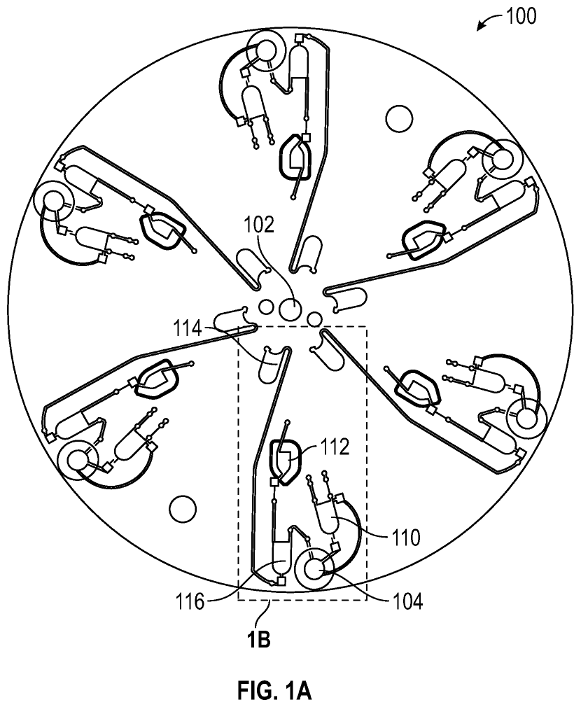

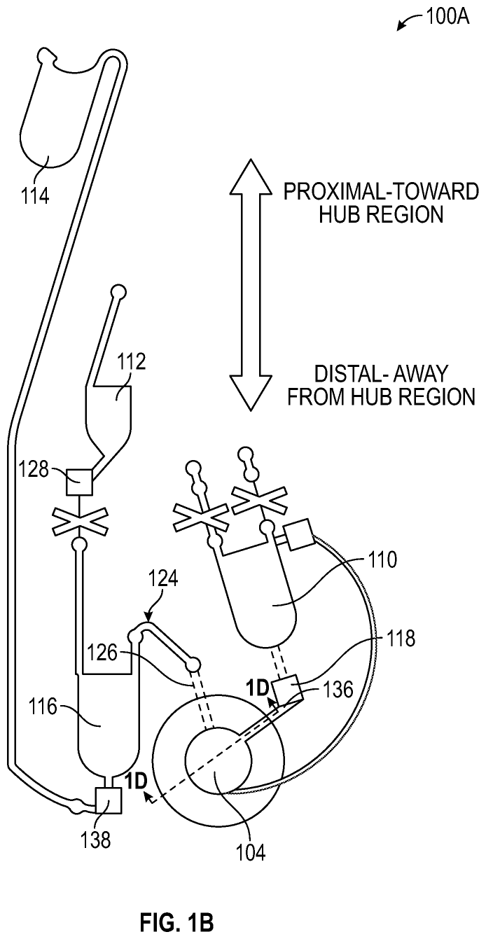

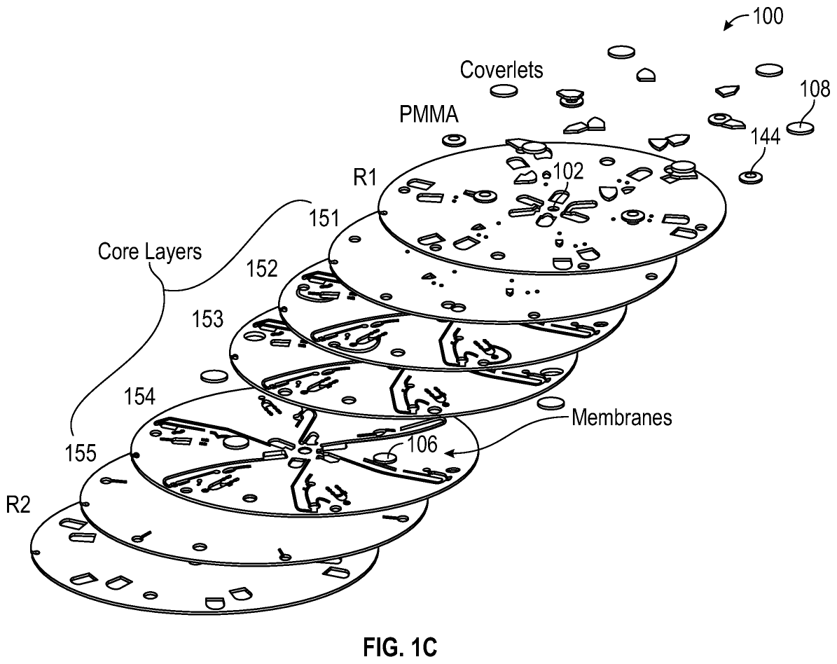

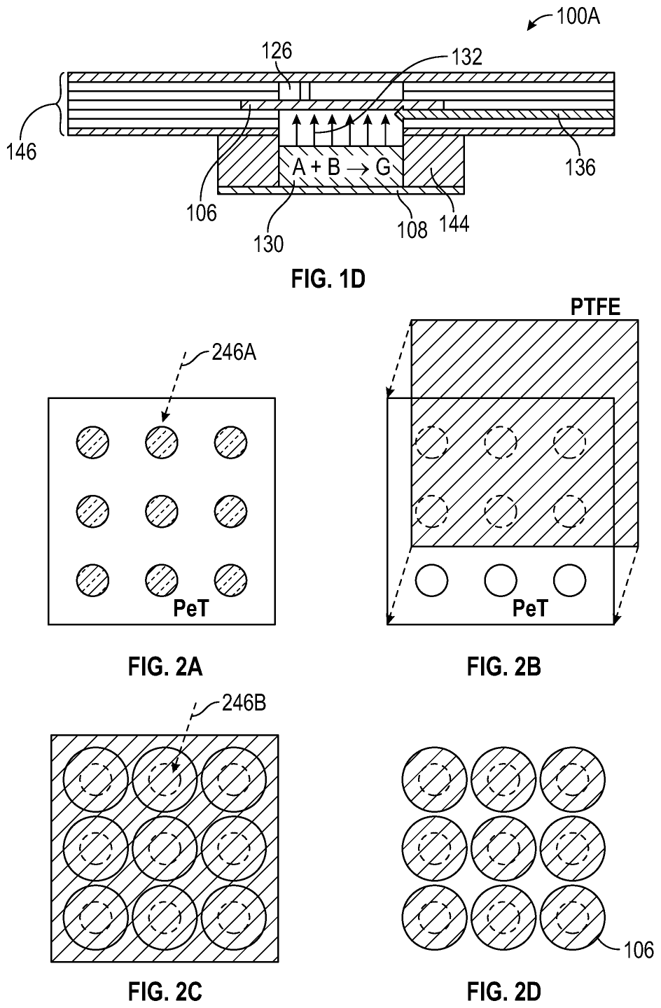

FIG. 1A illustrates generally an example of a microfluidic assembly 100 comprising a disc, the microfluidic assembly 100 configured to support inward fluidic displacement (IFD). The configuration of FIG. 1A includes a hub region 102 defining a center of rotation (CoR). A sample inlet region 112 can receive a sample, such as a swab (or a portion of a swab) holding a liquid sample. A liquid reagent chamber 110 can house a liquid reagent, and a dry reagent region 104 can house a dry reagent. Rotation of the microfluidic assembly 100 about the CoR at the hub region 102 can drive liquid from the sample inlet region 112 to a sample chamber 116 (with sample elute motion in a direction extending distally outward from the hub region 102). Mixing of a liquid reagent from the liquid reagent chamber 110 and a dry reagent 104 in the dry reagent region can combine, such as forming a gas-evolving reaction. Gas evolved from the reaction can drive sample elute from the sample chamber 116 to a sample recovery chamber 114, such as for further processing. In an example, the sample inlet region 112 can receive a lysate comprising a cellular medium.

FIG. 1B illustrates generally an example of a portion 100A of the microfluidic assembly of FIG. 1A. In the portion 100A shown in FIG. 1B, a sample eluted from a swab or other sample holder in the sample inlet region 112 can be transferred to a sample chamber 116, such as through a sample inlet valve 128, such as driven by rotation of the microfluidic assembly. Generally, valve structures such as the sample inlet valve 128 or a liquid reagent chamber valve 118 can be used to selective fluidically couple chambers or regions of the microfluidic assembly to other portions of the microfluidic assembly. A liquid reagent chamber 110 can house a liquid reagent (such as water or an acid). For example, the liquid reagent chamber valve 118 can fluidically couple the liquid reagent chamber to a dry reagent region 104, such as in a selective manner actuated using optical energy (e.g., a laser). The dry reagent region 104 can include one or more dry reagents, such as a combination (e.g., a mixture) of a dry acid and a dry base. Actuation of the valve 118 can allow the liquid reagent from the liquid reagent chamber 110 to flow into the dry reagent region 104 (with other channels coupled to the liquid reagent chamber 110 sealed as indicated by “X” symbols), such as driven by rotation of the microfluidic assembly. Gas evolved from an acid-base neutralization reaction in the dry reagent region 104 can be directed to the sample chamber 116 through a gas transfer channel 116. A fluidic channel between the sample inlet region 112 and the sample chamber 116 can be sealed, and gas evolved from the acid-base neutralization reaction can propel sample elute from the sample chamber 116 to a sample recovery chamber 114 for further processing downstream. The sample recovery chamber 114 can be located more proximally to a hub region of the microfluidic device as compared to the sample chamber 116, as shown in FIG. 1A and other examples.

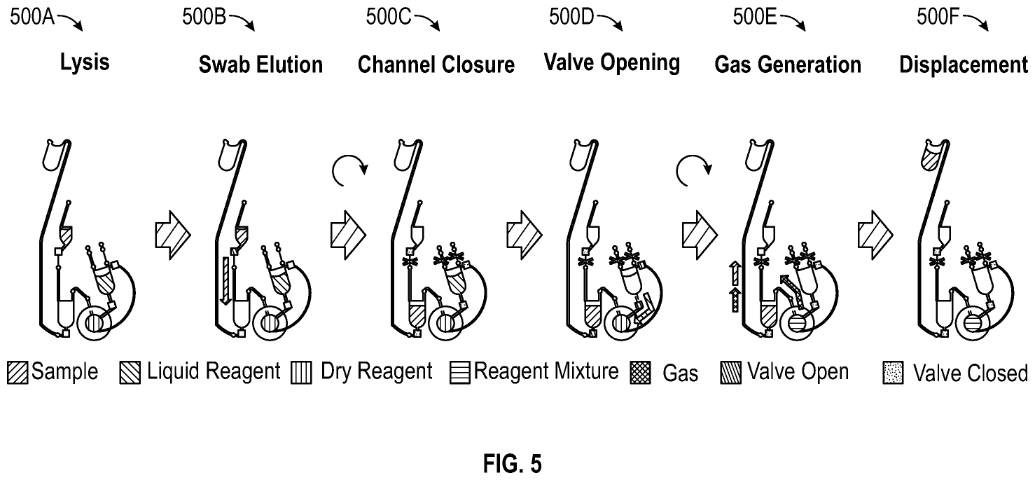

Generally, using the approach and configuration shown and described in relation to FIG. 1A and the examples below (e.g., FIG. 1B, FIG. 1C, FIG. 1D, FIG. 5, inward fluid displacement (IFD) can be rapidly driven (˜2 seconds) by gas generated on-board without requiring hands-on manipulation, highly corrosive reagents, or bulky peripheral equipment. Such an approach does not require manual disc manipulation and can use, for example, active valving configurations for automatable fluid delivery, neutralization reaction activation, and leak-free vent and inlet sealing, as shown and described below. Generally, reagents for the gas-evolving reaction used to drive IFD may be stored stably on-disc for months (e.g., six months or longer), highlighting the robust nature of this approach and its potential for field-forward use. Generally, the configuration of FIG. 1A (and discussed in further detail in relation to other examples below) is compatible with NAATs, including “gold standard” polymerase chain reaction (PCR) methods and loop-mediated isothermal amplification (LAMP), when used to provide CO₂-driven IFD. On-disc integration of NAATs with an upstream IFD strategy (such as using the configuration shown in FIG. 1A, FIG. 1B, FIG. 1C, FIG. 1D, and FIG. 5) can facilitate implementation of sample-to-answer genetic analysis lab-on-a-disc (LoaD) devices.

FIG. 1C illustrates generally an exploded view of layers and other structures that can form the microfluidic assembly 100 of FIG. 1A. A disc assembly (comprising layers R1, R2, and core layers 151, 152, 153, 154, and 155) can form a seven-layer planar assembly. The microfluidic assembly 100 can be fabricated at least in part using a ‘print-cut-laminate’ sequence. For example, the structure shown in illustrative example of FIG. 1C was captured in AutoCAD software (2019, Autodesk, Inc. San Rafael, CA) and was fabricated by cutting polyethylene terephthalate (PeT) films (Film Source, Inc., Maryland Heights, MO) using a CO2 laser (VLS3.50, Universal® Laser Systems, Scottsdale, AZ). The microfluidic assembly 100 of FIG. 1C comprises five core thermoplastic layers (core layers 151, 152, 153, 154, and 155), which housed the majority of the device structure, and two “reinforcing layers” (R1, R2) to improve valving performance. Following alignment of the five core layers 151, 152, 153, 154, and 155, circular, laser-cut polytetrafluoroethylene (PTFE) membranes (10.00 millimeter radius, such as a membrane 106) were nested into cutouts in the central layer (core layer 153).

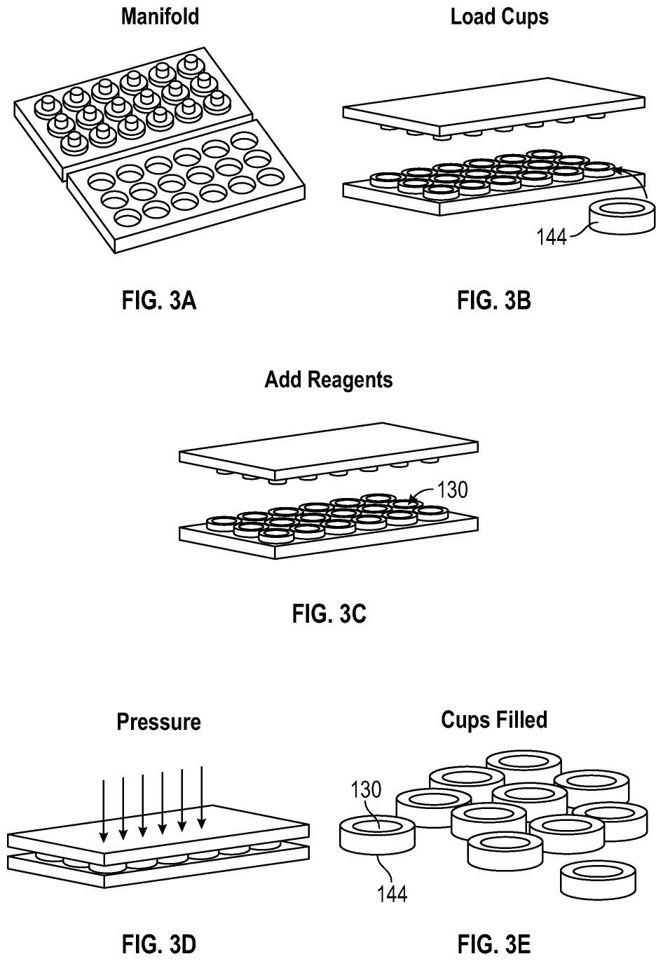

Device lamination (UltraLam, 250B, Akiles Products, Inc., Mira Loma, CA) was used to activate heat-sensitive adhesive (HSA, EL-7970-39, Adhesives Research, Inc., Glen Rock, PA) coating the primary fluidic layers (core layers 152 and 154), effectively bonding PeT layers together and anchoring PTFE membranes. The black PeT (core layer 153) (Lumirror* X30, Toray Industries, Inc., Chuo-ku, Tokyo, Japan) separating the fluidic layers enabled laser-actuated valving. Reinforcing layers (R1, R2), comprising PeT with HSA on one side, were attached to either side of the device via a second lamination step. Polymethyl methacrylate (PMMA) (1.5 mm thickness, McMaster Carr, Elmhurst, IL) cups, such as a cup 144, were loaded with dry reagents for on-board storage using a custom 3-D printed press (Form 3 printer and RS-F2-GPCL-04 resin, FormLabs, Somerville, MA, with the technique discussed further below in relation to FIG. 3A, FIG. 3B, FIG. 3C, FIG. 3D, and FIG. 3E). Up to 18 cups were placed into wells in the bottom half of the press and filled with sodium bicarbonate (Thermo Fisher Scientific, Waltham, MA) or a sodium bicarbonate-citric acid mixture. In some examples, citric acid (Sigma-Aldrich, Inc. St. Louis, MO) was mixed with the sodium bicarbonate prior to cup loading such that each contained, on average, 3 mg citric acid and 527 mg sodium bicarbonate. The press top was then aligned with the bottom component, and pressure was applied to compress reagents within each cup. Reagent cups and PMMA components for swab acceptance (0.5 mm thickness, Astra Products, Copiague, NY), were affixed to the device using pressure sensitive adhesive (PSA, Arcare 7876, Adhesives Research Inc.). In an example, to enable recovery and off-disc post-processing of displaced lysates, sample recovery chambers were augmented by PMMA (McMaster Carr) capped with PTFE membranes (shown as coverlets, including coverlet 108 in FIG. 1C).

FIG. 1D illustrates a section view of a portion 100A of the microfluidic assembly of FIG. 1A. A seven-layer planar assembly 146, as shown and described above in FIG. 1C, can be mechanically attached to one or more dry reagent cups (such as a cup 144 shown in FIG. 1C and FIG. 1D). The cup 144 can be capped with a lid 108 or membrane, to a keep a dry reagent 130 captive. When mixed with a liquid reagent 136, the dry reagent 130 can react to evolve gas 132. The gas 132 can permeate a gas-permeable membrane 106. The gas 132 can be directed to other portions of the microfluidic assembly, such as through a gas transfer channel 126 defined by or included as a portion of the microfluidic assembly. As discussed elsewhere herein, such gas evolution can be used to provide inward fluid displacement (IFD). Various reagents can be used. For example, a combination of a dry acid, “A,” and a dry base, “B,” can be used as the dry reagent 130. The liquid reagent 136 can be water, such as activating an acid-base neutralization reaction (A+B) to form a gas 132, “G.” The acid-base neutralization reaction can evolve CO₂, which can be used for liquid propulsion (e.g., inward fluid displacement) without risk of sample contamination or otherwise precluding NAAT or other downstream processing of a sample propelled by such gas.

FIG. 2A, FIG. 2B, FIG. 2C, and FIG. 2D illustrate various operations that can be used to fabricate a membrane structure (e.g., a gas permeable membrane) or other portion of the microfluidic assembly of FIG. 1A. For example, at FIG. 2A, a beam 246A from a CO₂ laser can be used to cut circular holes (2.5 mm radius) into a PeT film. In FIG. 2B, a PTFE membrane is aligned, and solvent bonded to the pre-cut PeT film from FIG. 2A. For example, upon alignment of the materials, the PTFE is wetted with methanol and allowed to dry at room temperature. In FIG. 2C, using a beam 246B from CO₂ laser, larger circular holes (5 mm radius) are cut into the bonded PTFE-PeT assembly. In FIG. 2D, flashing (excess material) is removed, revealing the PeT supported inserts. Prior to lamination, the PTFE inserts are nested into full thickness cutouts within the central layer of the disc (shown as membrane 106 applied to core layer 153 in FIG. 1C, with PTFE side up).

FIG. 3A, FIG. 3B, FIG. 3C, FIG. 3D, and FIG. 3E show operations that can be used to provide a dry reagent region (e.g., a cup), such as can be packed with one or more dry reagents that can be used for gas generation. FIG. 3A shows a 3D-printed manifold used to facilitate uniform loading of PMMA dry reagent cups. FIG. 3B shows PMMA cups (such as a cup 144) nested into recessed wells in the bottom manifold plate. FIG. 3C shows the cups covered with solid dry reagent 130 before the excess was scraped away with the flat edge of the manifold top, such that each cup was filled evenly and to the brim. FIG. 3D shows the top manifold plate aligned to position its cylindrical protrusions above the cup openings, then pressure is applied. FIG. 3E shows respective filled cups removed from the manifold, such as a cup 144 containing the dry reagent 130. Such cups can be applied to a disc-shaped microfluidic structure to provide the dry reagent region mentioned in relation to other examples in this document.

Evolution of carbon dioxide (CO₂) gas via acid-base neutralization is used for the IFD approach. For example, in respective dry reagent regions, neutralization occurs within a polymethyl methacrylate (PMMA) reagent cup 144 affixed to the disc assembly above an embedded polytetrafluoroethylene (PTFE) membrane. The gas-permeable, hydrophobic PTFE membranes sequester the solid and liquid reagents, preventing direct contact with the sample solution while allowing CO₂ gas generated as a byproduct of the neutralization reaction to pass into a gas transfer channel, driving IFD. The approach shown in FIG. 3A, FIG. 3B, FIG. 3C, FIG. 3D, and FIG. 3E generally results in average reagent mass per cup with less variability between replicates as compared to other approaches. According to an illustrative example, using the press, 0.039±0.002 grams (coefficient of variation (CV)=5.92%) NaHCO₃ was loaded into each cup, relative to 0.030±0.003 g (CV=10.8%) delivered via then slurry technique (n=15 each). The added reagent mass provided greater potential for gas evolution and reduced the dead air volume within the reaction chambers, both of which stand to enhance IFD effectiveness. Dry compression of reagents permits storage of NaHCO₃-citric acid mixtures without neutralization initiation. This reagent storage approach could also be applied to other dry mixtures for on-disc gas generation, such as baking powder.

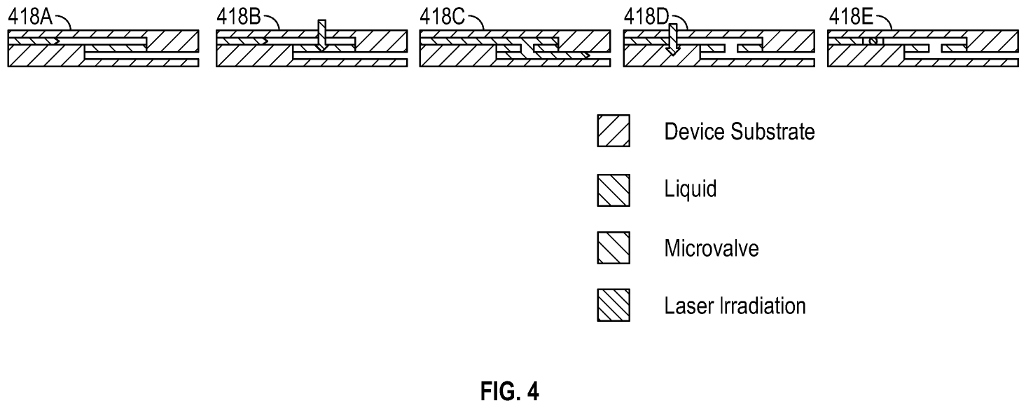

FIG. 4 illustrates a structure and related operation of a valve, such as can be included as a portion of the microfluidic assembly of FIG. 1A, and the detailed view shown in FIG. 1B and discussed in relation to other examples herein. The value configuration shown in FIG. 4 can be actuated using laser irradiation and can be re-sealed using laser irradiation. For example, at 418A, the valve is shown in a closed configuration, with the microvalve structure isolating a liquid at left from an outlet at right. At 418B, laser irradiation is applied, such as to breach the microvalve structure (e.g., causing pinholes or other apertures in a polymeric microvalve structure). At 418C, fluid can traverse the valve in the opened state after laser irradiation at 418B. Such a valve structure can be closed or re-sealed, such as by application of laser radiation at 418D such as to deform the valve material to occlude a liquid channel. At 418E, after re-sealing, the fluid at left is again isolated from the outlet at left.

Laser-actuation of valves in experimental examples described below were achieved using the Power, Time, and Adjustable Z-Height Laser (PrTZAL). Microdevice rotation and position relative to a 700 milliwatt (mW), 638 nanometer (nm) laser diode (L638P700 M, ThorLabs, Inc., Newton, NJ) were controlled by a brushless DC motor, photointerrupting optical switch (TT Electronics/Optek Technology, Woking, UK), and a motorized translational stage (MTS50-Z8, Thorlabs, Inc.). A z-height of the laser diode relative to the microdevice surface was adjusted between 15.00 mm for valve opening (500 mW, 500 milliseconds (ms)) and 27.00 mm for channel closures (700 mW, 2500 ms) using two stepper motors (Polulu Robotics and Electronics, Las Vegas, NV). All functions were controlled using a 32-bit multi-processing microcontroller (Propeller P8X32A-M44, Parallax, Inc., Rocklin, CA).

As mentioned above, because centrifugal force acts in radially-outward from the center of rotation (CoR), a count of possible sequential on-board unit operations is limited by the disc radius of the microfluidic assembly. Such geometric constraints can preclude total microfluidic sample-to-answer processing. The workflow shown FIG. 5 shows a combination of rotationally-driven and gas-evolution IFD, such as facilitating a greater count of on-board unit operations in a microfluidic device, as compared to a rotationally-driven approach, alone. In the approach shown in FIG. 5 and discussed in relation to other examples here, laser-based valving can be used, and can be superior to less robust approaches that exhibit failure (leaking) within pressurized systems or when alcohol-based solvents are needed. Active laser sealing helps to provide leak-free vent and inlet closure, while laser-based valve opening permits timed fluid release, including wetting of reagents stored on-disc, and therefore initiation of a gas evolving neutralization reaction.

As shown in the illustrative example of FIG. 1A, to establish proof-of-concept IFD performance and NAAT compatibility, six identical domains contained features that enabled on-board direct-from-swab enzymatic lysis and subsequent displacement of the recovered lysate back towards the CoR, and a detailed view of such a domain is shown in the portion 100A of the microfluidic device shown in FIG. 1B. FIG. 5 illustrates operations that can be performed, such as for sample extraction, using the microfluidic assembly of FIG. 1A and the structures shown in FIG. 1B.

As an illustrative example, referring to FIG. 5, a first on-disc unit operation at 500A in an IFD can include direct-from-swab enzymatic cellular lysis. Following completion of this step, lysate is eluted, such as from an absorbent substrate in a sample inlet region and moved to a downstream chamber. For example, at 500B, a laser-actuated valve (e.g. “laser valve”) beneath a swab chamber (sample inlet region) is opened and a lysate is centrifugally pumped into the sample chamber. Fluid recovery from absorbent substrates is generally related to placement of the swab chamber on the disc assembly. While placing the swab chamber closer to the device center can be advantageous in terms of maximizing downstream microfluidic “real estate,” a direct dependence of centrifugal force on the radial distance from the CoR (rotor length) can make this impractical. Experimentally, this was demonstrated by comparing the volumes of dye recovered from two sets of swab cuttings at varied distances from the CoR. To enhance fluid recovery, the disc assembly was rotated at 3000 rpm (120 s), corresponding to a limit of many spin systems. When placed as close as sterically permitted to CoR (14.2 mm), no observable fluid volume was eluted from the cotton substrate (142.9 g). Conversely, approximately 10 microliters (μL) of a 14 μL dye added to swab cuttings could be recovered from the substrate via centrifugation at a radial distance of 39.60 mm (398.5 g); this distance was used in all ensuing IFD experiments. On-disc lysis and subsequent IFD can be applied to other sample types, such as blood or saliva, without swab integration. Lysis from a sample on a solid substrate (e.g., swab) is, perhaps, a more challenging scenario as compared to other sample types.

After lysate elution from the swab, at 500 C, laser-sealing of the vent and inlet channels of the sample and acid chambers can be performed, such that a remaining open vent is associated with the sample recovery chamber. At 500D, laser valves associated with a liquid reagent outlet, liquid reagent vent, and sample chamber outlet can be opened. According, at 500E, gas produced by a neutralization reaction flows into the sample chamber, placing direct pneumatic pressure on the lysate. As pressure builds, lysate is driven towards the open channel, ultimately displacing both liquid and gas flow towards the CoR, into a sample recovery chamber as shown illustratively at 500 F. This IFD approach does not require working ‘displacer’ fluids to be manually loaded by the user immediately prior to assay initiation, and in the approach shown in FIG. 5, some or all reagents may be incorporated during device fabrication. Because IFD reagents in this approach can be stored at the edge of the device, they do not consume more valuable, centrally located surface area.

Two failure modes were noted during empirical evaluation. Incomplete channel closures resulted in a poorly sealed system that failed to produce sufficient gas pressure to drive IFD. This failure mode was remedied by using the PrTZAL system for automated alignment and channel closure. To test the strength of the channel closures, fully sealed acid-base neutralization reagent cups were filled and pressurized. However, the valve leading to the fluid transfer channel was not opened. The channel closures did not fail; rather, the failure point in these sealed, pressurized chambers was an adhesive bond (PSA) between the PMMA reagent cups and the PeT disc assembly surface. This issue was eliminated by ensuring PSA adhesion via overnight disc curing under constant weight and pressure (˜10 pounds). It should be noted that though a laser diode is used for flow control (e.g., laser valve actuation), inclusion of the laser diode did not increase the footprint of the mechatronic platform for LoaD control, and therefore does not compromise portability.

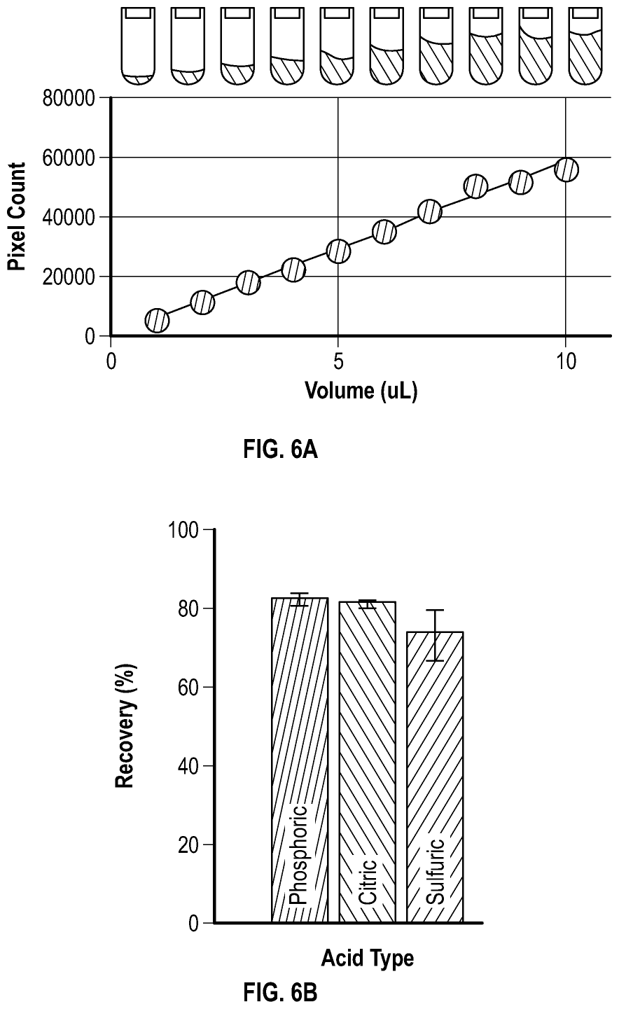

FIG. 6A shows an illustrative example of a correlation between extracted sample volume and pixel count for evaluation of performance of sample extraction assisted by gas evolution in a microfluidic assembly as shown in FIG. 1A and using the structures shown in FIG. 1B. For example, the portion above the plot shows images of aqueous green dye after disc loading, swab elution, and inward displacement to a sample recovery chamber, with the plot representing a calibration curve relating dye volume vs. pixel count, showing a linear correlation (R2=0.99). FIG. 6B shows corresponding experimentally-obtained sample recovery percentages (by volume) corresponding to different acid-base neutralization reactions for gas generation, using equimolar concentrations of phosphoric, citric, and sulfuric (each n=6).

Generally, to obtain the results shown in FIG. 6A, regions of interest (ROIs) encompassing the sample and sample recovery chambers were selected from images captured following swab elution and IFD, respectively. Within each cropped image, pixels attributable to the dye solution were specifically selected via pixel-based masking. Adjustment of color thresholds within the ImageJ freeware permitted selection of pixels containing dye, while excluding those associated with the image background and microdevice surfaces. Generally, within an image, the pixels associated with the dye solution were selected through application of color thresholds (hue: 80-255, saturation: 89-255, brightness: 60-191), then enumerated.

The calibration curve of FIG. 6A was used to determine the volumes of dye eluted from swab cuttings and dye present in the sample recovery chamber following IFD. As shown in FIG. 6B, a percentage of dye eluted from the swab and successfully displaced into the sample recovery chamber was comparable using equimolar concentrations sulfuric, phosphoric, and citric acids; the latter two are classified as weak acids. Weak acids can provide enhanced compatibility with device materials. The recoveries (approximately 80%) obtained from displacement using both weak acids were statistically similar (unpaired t-test, α=0.05, p-value=0.58); no detriment to displacement performance was observed relative to the strong acid. Citric acid is amenable to dry, on-disc storage. That is, dry citric acid mixed and stored with NaHCO₃ on-disc may be activated via simple rehydration with water. In this way, the user need not handle an acid directly and no liquid reagents must be stored on the disc. The dry, on-board citric acid-NaHCO₃ mixture was used in all subsequent IFD reactions for the experimentally-obtained results discussed below.

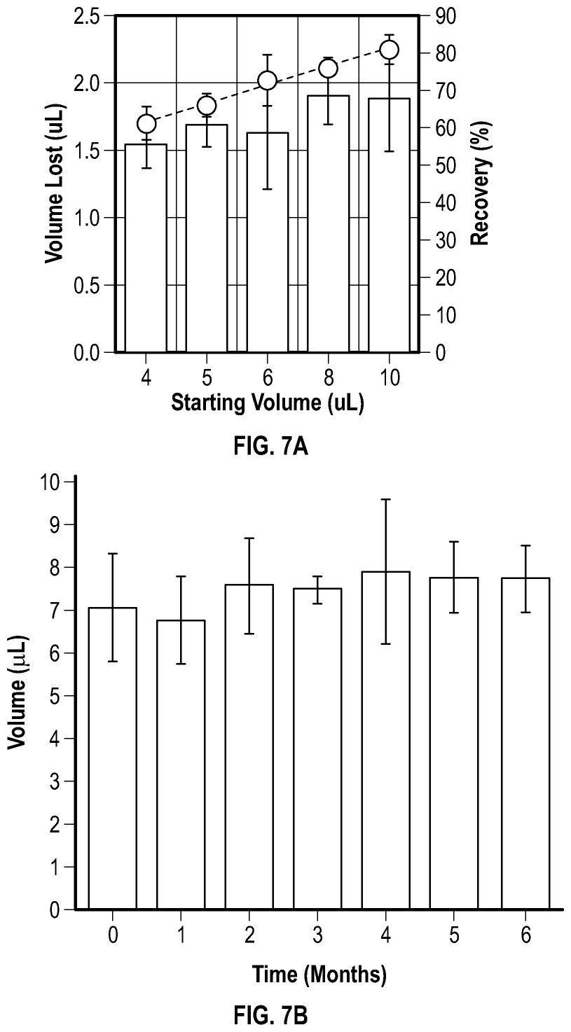

FIG. 7A shows an illustrative example of a correlation between extracted sample volume and starting sample volume for evaluation of performance of sample extraction assisted by gas evolution in a microfluidic assembly as shown in FIG. 1A and using the structures shown in FIG. 1B. Generally, to more thoroughly characterize fluid loss and recovery, IFD was performed using a range of aqueous dye volumes as shown illustratively in FIG. 7A. For each initial dye volume, calculated recoveries (%) following displacement are shown as dots and the mean volumes lost during IFD are shown as histogram bars (each n=3). The strong, positive linear correlation established between recovery and starting volume (R2=0.992) tracked with reproducible dye losses of 1.5-2 μL observed across conditions.

A single factor ANOVA indicated that these fluid losses are statistically similar across all initial dye volumes (α=0.05, p-value=0.51). Despite recovery rates below 100%, the sample chambers at the disc periphery were reproducibly visually empty; sufficient pneumatic pressure was generated to fully drive all fluid out of the sample chamber into the downstream architecture. Consequently, without being bound by theory, fluid losses were inferred not to be the result of incomplete IFD, but rather of limitations in the laser valving and PCL fabrication approaches. For example, normally-closed laser valve patches may retain small volumes of fluid at the egress point (0.5-2 μL); likewise, microscopic crevices (manufactured roughness) along the laser-ablated channel walls may retain fluid.

FIG. 7B shows an illustrative example of a correlation between extracted sample volume and aging of a dry reagent for evaluation of performance of sample extraction assisted by gas evolution in a microfluidic assembly as shown in FIG. 1A and using the structures shown in FIG. 1B. On-disc stability of the citric acid-NaHCO₃ mixture was experimentally evaluated over time. Seven discs, prepared concurrently, were tested using seven successive dye studies (IFD of 10 μL aqueous green dye) at one-month intervals (time points 0-6, n=4 each). Recovered volumes (calculated using image analysis in conjunction with the standard curve shown in FIG. 6A) were statistically similar across time according to a single factor ANOVA (α=0.05, p=0.69), as shown in FIG. 7B. Therefore, successful displacement in similar structures is believed achievable for at least six months after fabrication, with no loss in performance, using dry-stored reagents. These findings demonstrate the stability of these reagents on-board.

The following examples of experimentally-obtained results show that CO₂ evolved during displacement is not detrimental to downstream nucleic acid amplification tests (NAATs). Generally, acidification of the sample resulting from CO₂ conversion to carbonic acid at the gas-sample interface did not inhibit NA amplification. In the examples below, on-disc microfluidic enzymatic lysis was performed, and sample IFD using gas evolution, prior to off-disc post-processing. For the sake of simplicity, DNA isolation was achieved using one-step prepGEM chemistry; prepGEM lysates are directly compatible with the polymerase chain reaction (PCR), precluding the need for additional purification steps. To facilitate lysate retrieval from the disc, a gas-permeable PTFE membrane capping the PMMA sample recovery chamber was punctured with a pipette tip.

Epithelial cells were collected from anonymous, consenting donors in the form of deidentified buccal swabs. Cells were eluted in 300 μL 1× Tris-EDTA (Thermo Fisher Scientific) by rolling the swab against the walls of a 0.5 mL Eppendorf tube. Epithelial cells stained with the green fluorescent nucleic acid stain Syto-11 (0.2 μL) (Thermo Fisher Scientific) were visualized and quantified using hemocytometry and a Zeiss Axio microscope. Cotton swab cuttings (˜1/8) (Puritan Medical Products, Guilford, ME) were spiked with 1500 cells, then sealed into the on-disc swab chamber. A 14 μL aliquot of a lysis cocktail (12.46 μL water, 1.4 μL 10× blue buffer, 0.14 μL prepGEM (MicroGEM International, PLC., Charlottesville, VA) was added to the swab chamber and incubated (75° C., 300 s; 95° C., 60 s) using a clamped dual-Peltier system. In the dye studies discussed above, aqueous green dye (14 μL) was used to visually represent the cellular lysate.

The liquid reagent chamber was pre-loaded with 10 μL of sulfuric or phosphoric acid (both Thermo Fisher Scientific), or deionized water (used to rehydrate citric acid-sodium bicarbonate dry mixture). Regardless of acid type, 1.63×10⁻⁵ mol were used in each reaction. After cellular lysis, a laser valve beneath the swab chamber was opened to permit lysate elution (3000 rpm/398.5 g, 120s) into the sample chamber. This inlet, along with the vent and inlet associated with the acid chamber, was laser sealed. Normally-closed laser valves beneath the sample and acid chambers were then opened. Device rotation (2000 rpm/257.1 g, 2 s) drove fluid from the acid chamber into the chamber containing dried reagents, thus initiating on-board acid-base neutralization. Gas evolved during the neutralization reaction passed through the PTFE membrane and into the sample chamber. Successful inward displacement was characterized by the sample solution transfer from the sample chamber and into the sample recovery chamber. Displaced lysates were recovered for off-disc post-processing by puncturing the PTFE membrane with a pipette tip. When pertinent, device images were captured using an Epson Perfection V100 Photo desktop scanner (Seiko Epson Corporation, Suwa, Nagano Prefecture, Japan).

Relative DNA concentrations were established via real-time PCR (RT-PCR) targeting the TPOX locus. Each 20 μL reaction, prepared in 1× SensiFast Probe Lo-ROX One-Step Master Mix (Meridian Bioscience, Memphis, TN), contained forward and reverse primers (0.4 μM each), 0.1 μM probe, and 4 μL diluted lysate (1:4, water). Triplicate amplification reactions were performed using a QuantStudio 5 (Thermo Fisher). An initial denaturation step (95° C., 210 s) was followed by 40 cycles of denaturation (95° C., 5 s) and annealing (60° C., 30 s) and a 4° C. hold step. Fluorescence was monitored in the FAM channel; resultant cycle threshold (Ct) values were calculated using the threshold generated automatically by the QuantStudio™ Design & Analysis software (v1.5.0). Short tandem repeat (STR) profiles were generated following multiplexed PCR amplification of DNA from neat on-disc lysates using the PowerPlex 18D system (Promega, Madison, WI) according to manufacturer's instructions using 28 cycles. Amplicons were electrophoretically separated and fluorescently detected using an ABI 3130 Genetic Analyzer (Applied Biosystems, Grand Island, NY). Gene Marker HID software (v2.7.6) (SoftGenetics, State College, PA) was used to analyze resultant STR profiles.

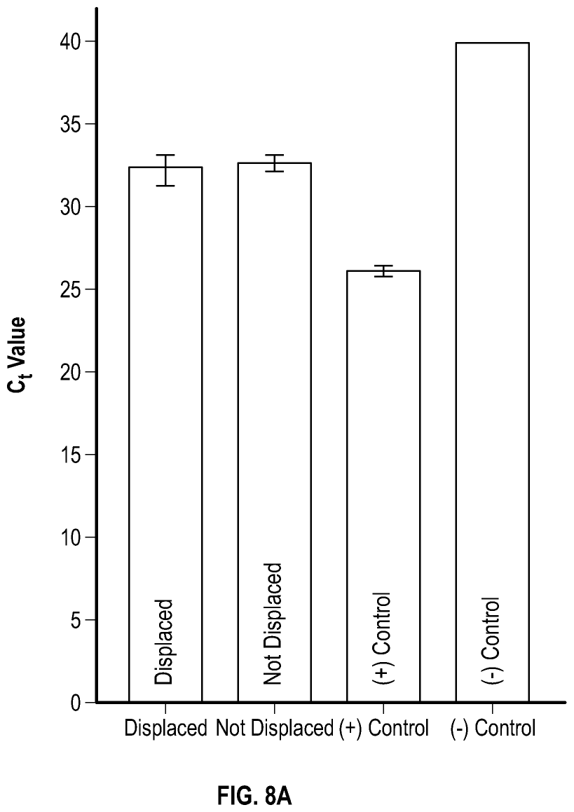

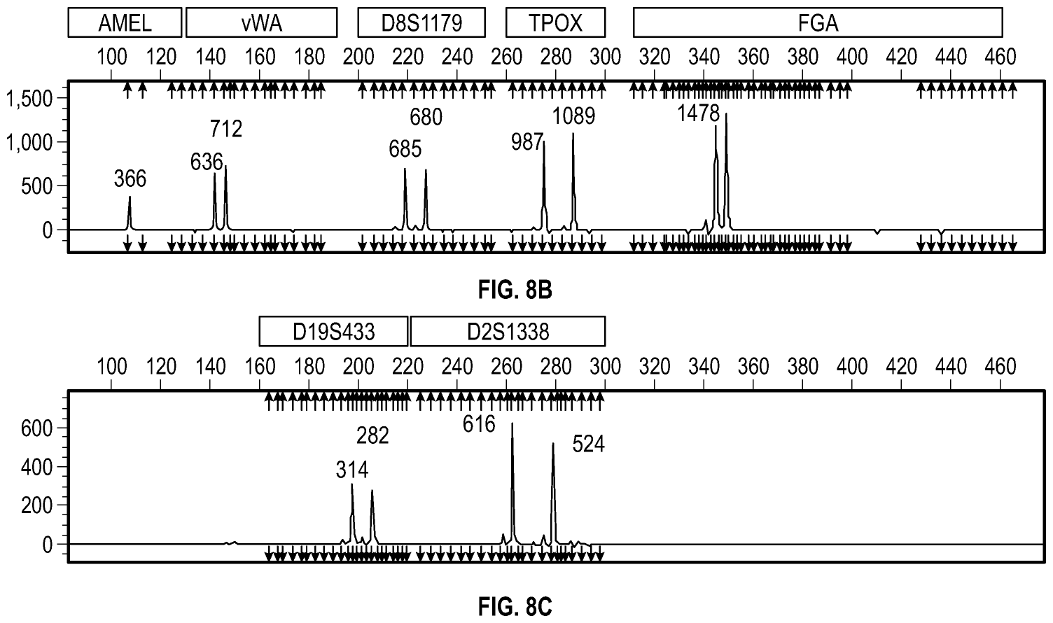

FIG. 8A shows an illustrative example of polymerase chain reaction (PCR) compatibility with a sample that has been displaced by gas evolution in a microfluidic assembly as shown in FIG. 1A and using the structures shown in FIG. 1B, versus a non-displaced sample, and controls. FIG. 8B and FIG. 8C show illustrative examples of full 18-plex short tandem repeat (STR) profiles obtained from DNA lysed and displaced by gas evolution in a microfluidic assembly as shown in FIG. 1A and using the structures shown in FIG. 1B prior to amplification.

DNA from recovered lysates was amplified via real-time PCR. Although commonly used for clinical applications (e.g., pathogen detection and human identification), real-time PCR may also be used as an analytical tool to evaluate the success of upstream nucleic acid preparation. Results of real-time PCR amplification of DNA from parallel on-disc, direct-from-swab cellular lysates with and without IFD produced comparable cycle threshold (Ct) values are shown in FIG. 8A. An unpaired t-test for difference between means indicated statistical similarity between Ct values across methods (α=0.05, p=0.62). If inhibition had resulted from gas exposure related to IFD, higher Ct values relative to the non-displaced samples would have been expected. Lysate compatibility with multiplexed PCR was also evaluated. Multiplexed PCR is generally more sensitive to inhibitors than real-time PCR, specifically through generation of short tandem repeat (STR) profiles. Briefly, in STR analysis, individual primer pairs target a number of distinct non-coding DNA regions, or loci, containing repeating nucleotide sequences (e.g., trimer, tetramers, or pentamers); at a given locus, a human individual may possess up to two polymorphic alleles of particular lengths (one inherited from each biological parent); a count of repeats may, coincidently, be the same length. FIG. 8B and FIG. 8C show two channels of a representative STR profile generated from a sample lysed and displaced on-disc. No signs of amplification inhibition were observed (‘ski slope’ effect); all expected peaks in the 18 loci probed were present with good balance within allele pairs (intralocus peak height balance). Accordingly, the real-time PCR results of FIG. 8A and STR results of FIG. 8B and FIG. 8C indicate that on-disc gas-driven displacement presents no observable detriment to downstream PCR amplification.

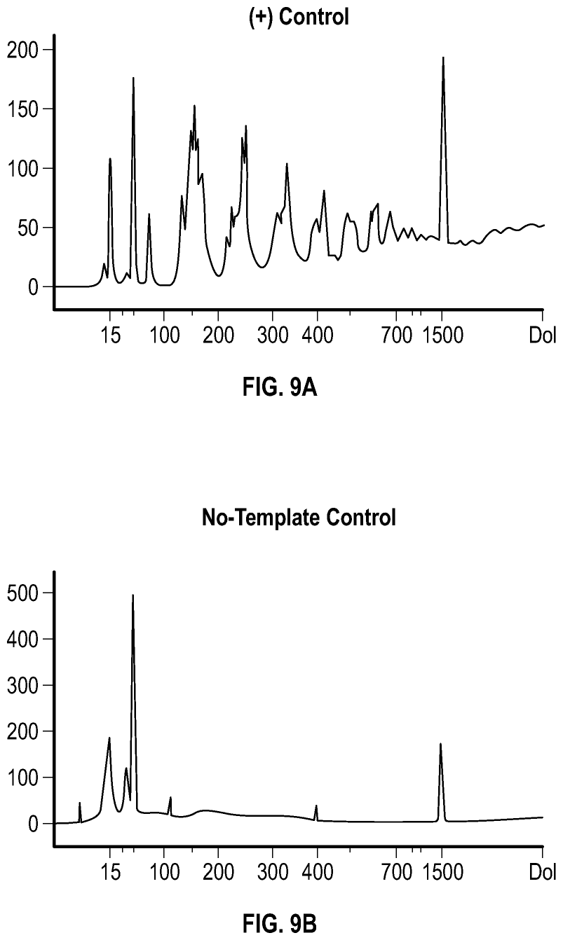

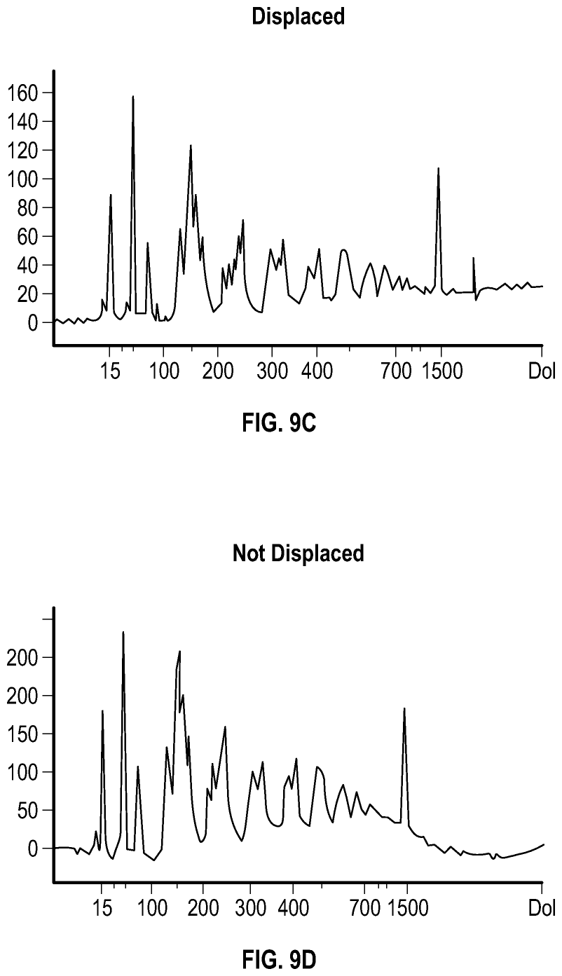

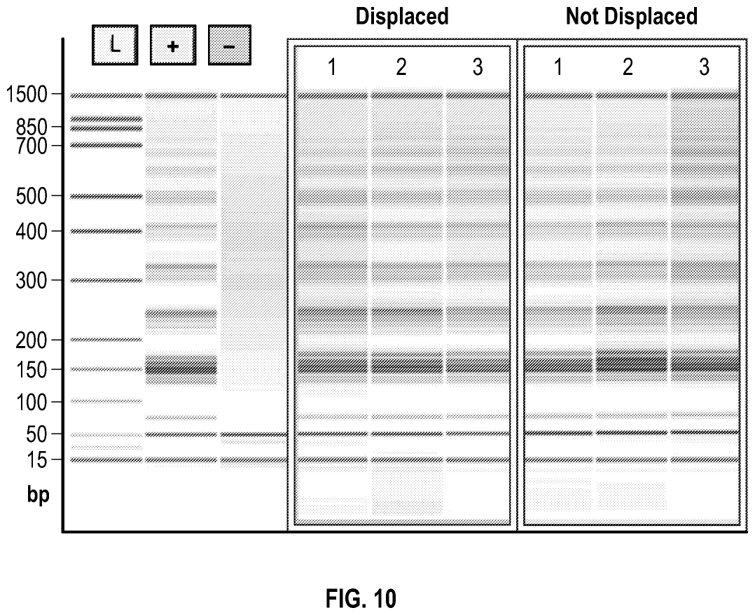

FIG. 9A, FIG. 9B. FIG. 9C, and FIG. 9D show illustrative examples comprising respective electropherograms of controls and samples obtained from DNA lysed and displaced by gas evolution in a microfluidic assembly as shown in FIG. 1A and using the structures shown in FIG. 1B. FIG. 10 shows illustrative examples of gel image renderings of triplicate samples, indicating successful amplification of samples displaced by gas evolution in a microfluidic assembly as shown in FIG. 1A and using the structures shown in FIG. 1B, versus controls.

PCR laboratory-based techniques are generally regarded as the gold standard NAATs. However, isothermal methods offer another approach, due in part, to simplified hardware configurations that ease implementation outside of traditional laboratory setting. Among these other approaches, loop-mediated isothermal amplification (LAMP) leverages a strand-displacing polymerase to facilitate exponential target amplification via repeated, sequential annealing and extension of 4-6 primers. Microchip electrophoretic separation of resultant polydisperse LAMP amplicons creates a repeating peak pattern over a substantial molecular range, as compared to a single peak observed following PCR. Such repeating patterns were apparent in experimentally-obtained electropherograms obtained from analysis of DNA amplified following on-disc lysis with and without IFD, shown respectively at FIG. 9C and FIG. 9D, as well as the positive control (commercial human genomic DNA), indicating successful, on-target amplification, shown in FIG. 9A. No peaks were observed in the no template control at FIG. 9B, indicating the absence of non-specific amplification.

Microchip electropherograms from triplicate on-disc DNA preparations with and without IFD were compiled. For ease of pattern recognition, these are converted to a ‘gel’ image rendering, where bands in each lane correspond to peaks in the original electropherograms (with such gel image renderings shown illustratively in FIG. 10). All sample lanes exhibited banding patterns indicative of successful LAMP, and the negative control (−) showed an absence of such banding, which clearly demonstrated reproducible isothermal amplification, with no detriment resulting from on-disc, gas-driven IFD.

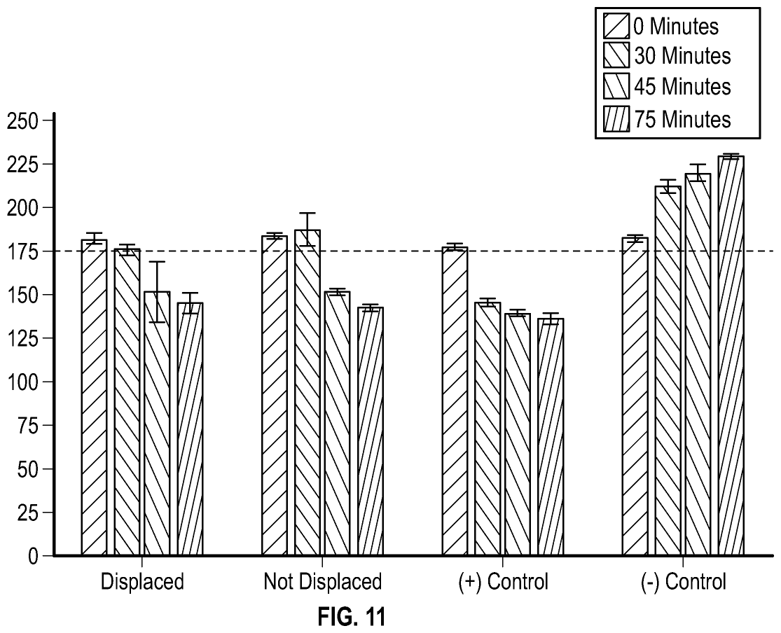

FIG. 11 shows illustrative examples colorimetric evaluation of LAMP-based detection on samples displaced by gas evolution in a microfluidic assembly as shown in FIG. 1A and using the structures shown in FIG. 1B, versus controls, and indicating successful detection of all samples in less than 45 minutes. The results of endpoint microchip electrophoresis detection shown in FIG. 10 are encouraging, but do not provide any insight regarding amplification speed. To achieve this, we supplemented the previous results with colorimetric reaction monitoring. LAMP compatibility with simple, visual detection facilitates such reaction monitoring. Incorporation of hydroxy naphthol blue (HNB), which changes from purple to blue with amplification, provided binary, semi-quantitative visual indication of target presence at discrete time points (0, 30, 45, and 75 minutes) in semi-real time. By 45 minutes, all samples appeared visibly blue (positive, as indicated by the hue value), while the no template control (“(−)”) was purple (negative). A threshold was established three standard deviations below the mean initial hue (0 min) across all samples (175.16 AU, indicated by the dashed line in FIG. 11); hue readings above (purple) and below (blue) this delineation indicated colorimetric negatives and positives, respectively. Hue analysis confirmed the naked eye visual results; by 45 min, the measured hue values of all samples were below the threshold, and therefore positive as shown illustratively in FIG. 11. Taken together, these colorimetric and electrophoretic results indicated that sample IFD did not hinder downstream LAMP. Generally, in the illustrative examples of FIG. 9A, FIG. 9B, FIG. 9C, FIG. 9D, FIG. 10, and FIG. 11, each reaction targeting the TPOX locus was comprised 0.2 μM F3 and B3, 0.8 μM LF and LB, and 1.6 μM FIP and BIP primers, 120 μM hydroxy naphthol blue (HNB), and 1.25 μL sample in 1× WarmStart (DNA and RNA) Master Mix (New England Biolabs, Ipswitch, MA). Images were captured using a smartphone (Huawei Technologies Co., Ltd. Shenzhen, China) after heating 0, 30, 45, and 75 min at 65° C. The hue of a 60-pixel diameter circular region of interest was measured from each tube at each timepoint using ImageJ. Endpoint electrophoretic amplicon separation and detection was performed using the DNA chip assay for the Bioanalyzer 2100 (Agilent Technologies, Santa Clara, CA).

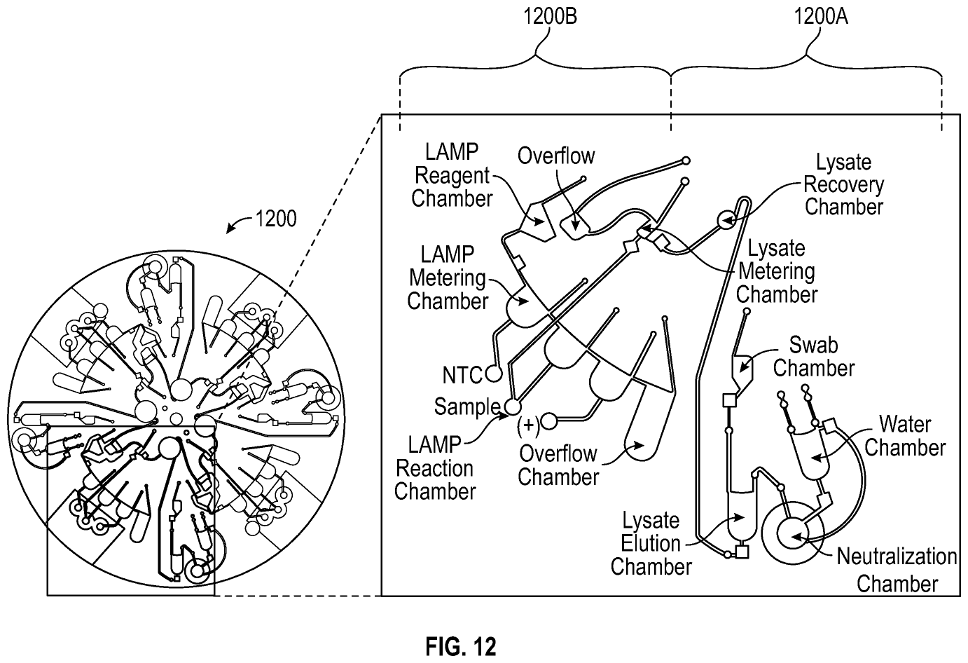

Generally, the examples of FIG. 9A, FIG. 9B, FIG. 9C, FIG. 9D, FIG. 10, and FIG. 11 used on-disc IFD, but off-disc downstream processing for performing LAMP. FIG. 12 illustrates generally an example of a microfluidic assembly 1200 comprising a disc, the microfluidic assembly 1200 configured to support inward fluidic displacement (IFD) for sample extraction along with downstream LAMP co-integrated on-disc. Generally, the microfluidic assembly 1200 can include features similar to the IFD sample-extraction features of the microfluidic assembly 100 of FIG. 1A and related examples, such as in a first region 1200A. In addition, the microfluidic assembly 1200 can include a second region 1200B defining or otherwise including features to perform LAMP, such as can include no template control (NTC) and positive control (“(+)”) indicating regions.

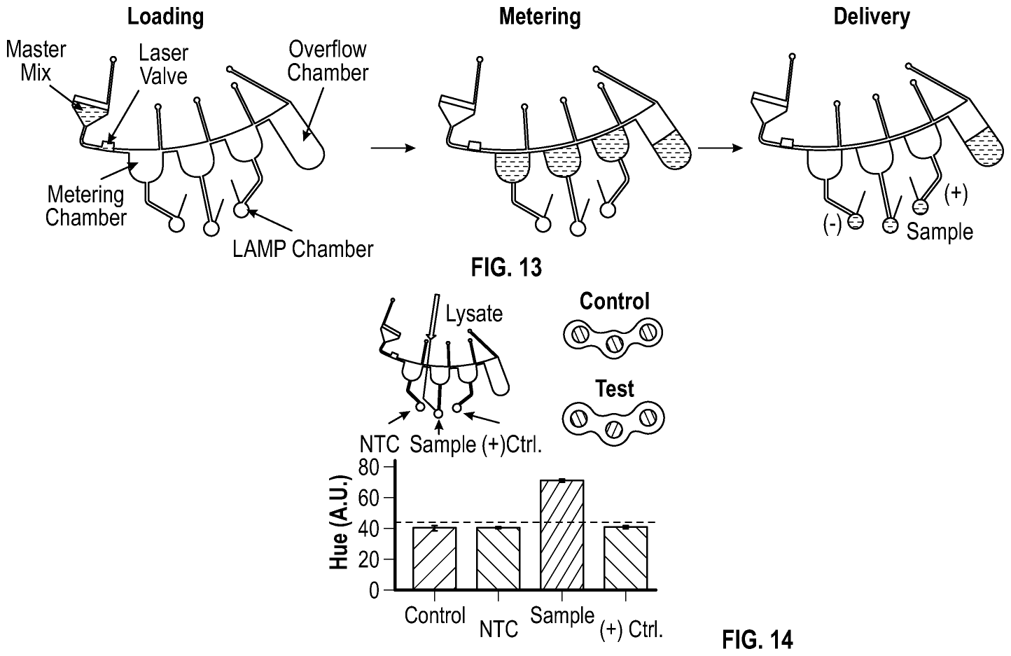

For example, FIG. 13 illustrates generally an example comprising operations to perform LAMP, such as using a microfluidic assembly 1200 as shown in FIG. 12, using features from the second region 1200A of FIG. 12. A LAMP master mix can be loaded into a chamber with a valve at the outlet, such as shown at “i.” and after valve actuation, respective aliquots can be transferred (such as using low-speed rotation of the disc) to respective metering chambers at “ii.” Higher-speed rotation can be used to deliver the metered aliquots to respective LAMP reaction chambers to provide respective indicators. In the example at “iii.” three chambers are shown, such as providing contemporaneous amplification of the sample (center location), no template control (“(−)”) and a positive control (“(+)”).

As a further illustration, FIG. 14 illustrates generally an example showing proper delivery of a lysate to the center sample chamber (indicated by hue), while such a lysate is isolated from the NTC and (+) control chambers, such as using the operations shown in FIG. 13. For example, the higher-speed rotation to transfer the sample aliquot of the LAMP master mix to the sample reaction chamber can also drive lysate to the sample reaction chamber. FIG. 14 illustrates that in a control case, with no lysate, all three reaction chambers indicate the same hue, whereas in the test case (where sample lysate is present) indicates a different hue for the center chamber, but the same hue for each of the controls. Such a result shows that contamination of controls by lysate is not occurring.

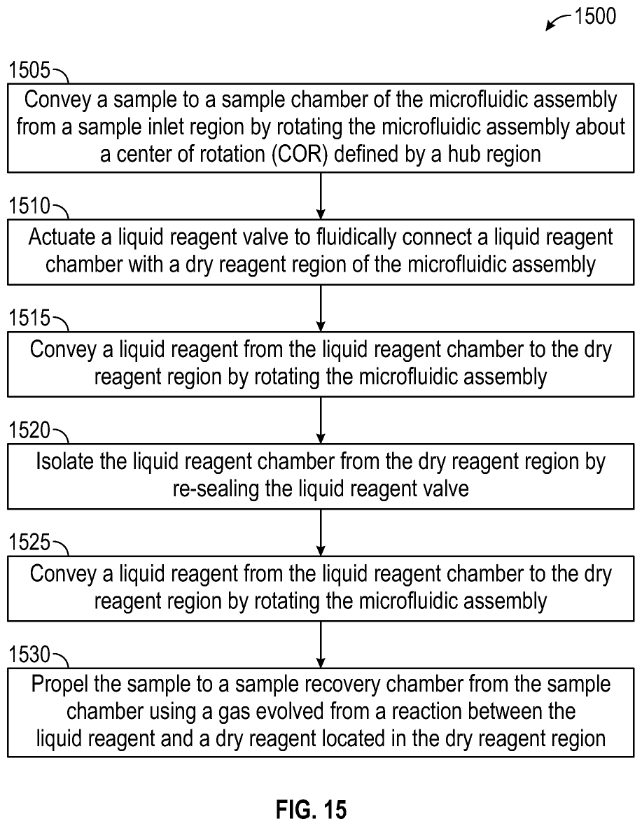

FIG. 15 illustrates generally a technique 1500, such as an automated (e.g., processor-controlled or otherwise machine-controlled) or semi-automated method, for propelling a sample to a sample recovery chamber, such as in a manner toward a center of rotation (CoR) using gas evolution. At 1505, a sample (such as a lysate) can be conveyed to a sample chamber of the microfluidic assembly from a sample inlet region by rotating the microfluidic assembly about a center of rotation (COR) defined by a hub region. At 1510, a liquid reagent valve can be actuated (e.g., open using laser irradiation) to fluidically connect a liquid reagent chamber with a dry reagent region of the microfluidic assembly. At 1515, a liquid reagent from the liquid reagent chamber can be conveyed to the dry reagent region by rotating the microfluidic assembly. At 1520, the liquid reagent chamber can be isolated from the dry reagent region by re-sealing the liquid reagent valve. At 1525, a liquid reagent can be conveyed from the liquid reagent chamber to the dry reagent region by rotating the microfluidic assembly. At 1530, the sample can be propelled to a sample recovery chamber from the sample chamber using a gas evolved from a reaction between the liquid reagent and a dry reagent located in the dry reagent region.

### Various Notes

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc., are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A microfluidic assembly configured for rotationally-driven operation, the microfluidic assembly comprising:
a hub region defining a center-of-rotation (CoR).
a dry reagent region;
a liquid reagent chamber fluidically isolated from the dry reagent region by a liquid reagent valve, the liquid reagent chamber located more proximally to the CoR as compared to the dry reagent region;
a sample chamber fluidically coupled with the dry reagent region through a gas transfer channel; and
a sample recovery chamber fluidically coupled with the sample chamber, the sample recovery chamber located more proximally to the CoR as compared to the dry reagent region and the sample chamber;
wherein the liquid reagent valve, when opened, permits a liquid reagent from the liquid reagent to flow in a direction distally with respect to the CoR to the dry reagent region in response to rotation of the microfluidic assembly about the CoR; and
wherein the gas transfer channel is configured to convey a gas evolved from a reaction between the liquid reagent and a dry reagent in the dry reagent region to the sample chamber to propel at least a portion of a sample in the sample chamber to the sample recovery chamber.

2. The microfluidic assembly of claim 1, wherein the liquid reagent valve is sealable to inhibit back-flow of the gas to the liquid reagent chamber.

3. The microfluidic assembly of claim 1, comprising a sample inlet region configured to receive the sample; and
a sample inlet valve located between the sample inlet region and the sample chamber.

4. The microfluidic assembly of claim 3, wherein the sample inlet region is sized and shaped to receive a swab eluting the sample comprising cellular media.

5. The microfluidic assembly of claim 4, wherein the sample inlet region is treated with or fluidically coupled to an enzyme for performing cellular lysis of the sample.

6. The microfluidic assembly of claim 3, wherein the sample inlet valve, when opened, permits the sample to flow to the sample chamber in response to rotation of the microfluidic assembly about the CoR.

7. The microfluidic assembly of claim 1, comprising a sample chamber outlet valve located between the sample recovery chamber and the sample chamber; and
wherein the fluidic coupling between the sample recovery chamber and the sample chamber is controlled by the sample chamber outlet valve.

8. The microfluidic assembly of claim 7, further comprising the liquid reagent; and
wherein the dry reagent and the liquid reagent, when mixed, establish the reaction comprising an acid-base neutralization reaction.

9. The microfluidic assembly of claim 1, wherein the dry reagent comprises a solid-phase mixture of an acidic compound and an alkaline compound.

10. The microfluidic assembly of claim 1, wherein the gas does not suppress or inhibit function of nucleic acid amplification test (NAAT) reagents downstream from the sample recovery chamber.

11. The microfluidic assembly of claim 10, comprising downstream structures including chambers for performing loop-mediated isothermal amplification (LAMP) on the sample, the downstream structures fluidically coupled with the sample recovery chamber.

12. The microfluidic assembly of claim 1, wherein the sample chamber, sample recovery chamber, liquid reagent chamber, and gas transfer channel are defined by or included as a portion of a planar multi-layer disc assembly.

13. The microfluidic assembly of claim 12, wherein the dry reagent region comprises a cavity defined by a structure separate from the multi-layer disc assembly.

14. The microfluidic assembly of claim 13, wherein the structure separate from the multi-layer disc assembly comprises a cup defining the cavity; and
wherein the dry reagent comprises a compressed mass in the cavity.

15. The microfluidic assembly of claim 14, comprising a gas-permeable membrane between the dry reagent and the gas transfer channel.

16. The microfluidic assembly of claim 1, wherein the liquid reagent valve is configured to be at least one of opened or sealed in response to irradiation by a laser.

17. A method for performing processing of sample using a rotationally-driven microfluidic assembly, the method comprising:
conveying a sample to a sample chamber of the microfluidic assembly from a sample inlet region by rotating the microfluidic assembly about a center of rotation (CoR) defined by a hub region;
actuating a liquid reagent valve to fluidically connect a liquid reagent chamber with a dry reagent region of the microfluidic assembly;
conveying a liquid reagent from the liquid reagent chamber to the dry reagent region by rotating the microfluidic assembly about the CoR;
isolating the liquid reagent chamber from the dry reagent region by re-sealing the liquid reagent valve; and
propelling the sample to a sample recovery chamber from the sample chamber using a gas evolved from a reaction between the liquid reagent and a dry reagent located in the dry reagent region, the gas conveyed to the sample chamber using a gas transfer channel from the dry reagent region;
wherein the sample recovery chamber is located more proximally to the CoR as compared to the dry reagent region and the sample chamber; and
wherein the sample is propelled at least in part inwardly toward the CoR by the gas.

18. The method of claim 17, comprising sealing the liquid reagent valve to inhibit back-flow of the gas to the liquid reagent chamber.

19. The method of claim 18, comprising fluidically isolating the sample chamber from the sample inlet region using a sample inlet valve, after elution of the sample comprising cellular media from a swab placed in the sample inlet region, and after conveying the sample to the sample chamber.

20. The method of claim 17, wherein the reaction comprises an acid-base neutralization reaction that occurs when the liquid reagent is mixed with the dry reagent.

21. The method of claim 17, wherein the dry reagent comprises a solid-phase mixture of an acidic compound and an alkaline compound.

22. The method of claim 17, wherein the gas does not suppress or inhibit function of nucleic acid amplification test (NAAT) reagents downstream from the sample recovery chamber.

23. The method of claim 22, comprising performing loop-mediated isothermal amplification (LAMP) on the sample using downstream structures fluidically coupled with the sample recovery chamber.

24. The method of claim 17, comprising sequestering a mixture of the liquid reagent and dry reagent in the dry reagent region from the gas transfer channel using a gas-permeable membrane.

25. The method of claim 17, wherein actuating the liquid reagent valve comprises irradiating the liquid reagent valve with a laser.
